(19) United States
(12) Patent Application Publication
(10) Pub. No.: US 2024/0298591 A1
(43) Pub. Date: Sep. 12, 2024

(54) Method for improving induction rate of luffa embryoid by radiation mutagenesis

(71) Applicants: Taizhou Institute of Agricultural Sciences of JAAS, Taizhou City (CN); Taizhou Institute of Research of NAU, Taizhou City (CN)

(72) Inventors: Cui FENG, Taizhou City (CN); Huiying LIU, Taizhou City (CN); Ru JI, Taizhou City (CN); Chuntao QIAN, Taizhou City (CN); Wei QIAN, Taizhou City (CN); Zhengwei YI, Taizhou City (CN); Weichen NI, Taizhou City (CN); Mingli YONG, Taizhou City (CN); Yingna FENG, Taizhou City (CN); Xiaojun SU, Taizhou City (CN)

(21) Appl. No.: 18/598,280

(22) Filed: Mar. 7, 2024

(30) Foreign Application Priority Data
Mar. 7, 2023 (CN) 202310208723.X

(51) Int. Cl. A01H 1/06; A01H 6/34

(52) U.S. Cl. CPC A01H 1/06; A01H 6/34

(57) ABSTRACT

The present disclosure provides a method for improving an induction rate of a luffa embryoid by radiation mutagenesis. The present disclosure belongs to the technical field of crop tissue culture and breeding. In the present disclosure, the method includes conducting radiation of pollens, conducting pollination of radiated pollens, conducting disinfection, and conducting cultivation. The method of the present disclosure provides a technical support for luffa haploid breeding and further improves a breeding efficiency.

### CROSS REFERENCE TO RELATED APPLICATION

This patent application claims the benefit and priority of Chinese Patent Application No. 202310208723.X, filed with the China National Intellectual Property Administration on Mar. 7, 2023, the disclosure of which is incorporated by reference herein in its entirety as part of the present application.

### TECHNICAL FIELD

The present disclosure relates to the technical field of crop tissue culture and breeding, and more specifically to a method for improving an induction rate of a luffa embryoid by radiation mutagenesis.

### BACKGROUND

Luffa belongs to the genus Luffa of the family Cucurbitaceae and is divided into ordinary and ribbed luffas. Luffa shows strong adaptability and is widely cultivated in temperate and tropical regions of the world. The luffa is also commonly cultivated in southern and northern China as one of the important melon vegetables in China. Luffa is rich in nutritional value, with its flesh being delicate and smooth and tasting delicious. In addition to being rich in protein, fat, crude fiber, sugar, vitamin B, vitamin C, xylan gum, calcium, phosphorus, iron and other minerals, the luffa also contains several major types of compounds such as sterol triterpenes, saponins, flavonoids, and phenols, as well as a variety of chemical monomers and other medicinal ingredients. As a vegetable used both as medicine and food, the luffa is deeply loved by people and has broad production and application prospects, such that its cultivation area has been expanding in recent years.

Luffa is a cross-pollinated crop, and its traditional breeding is achieved mainly through self-crossing or cross-breeding. However, these approaches show problems such as long breeding cycles and self-incompatibility, and cannot meet the current rapid demand for new varieties. In order to quickly solve the first-generation breeding method of hybrids, there are natural haploid doubling or artificial doubling techniques that can be used. However, there is an extremely low frequency of spontaneous generation for haploids in melon crops. Common methods for inducing haploid plants include anther culture, unfertilized ovary induction, and radiation pollen induction. At present, haploids have been obtained for five genera, including Cucurbita, Lagenaria, Citrullus, and Momordica, and some of which have been applied in breeding. However, there are few reports on luffa haploid technology mainly due to the difficulty in embryoid body induction and an extremely low induction rate of only 0.1%.

In summary, it is an urgent problem that needs to be solved by those skilled in the art to provide a method for improving an induction rate of a luffa embryoid by radiation mutagenesis.

### SUMMARY OF THE INVENTION

In view of this, the present disclosure provides a method for improving an induction rate of a luffa embryoid by radiation mutagenesis. In the present disclosure, the method mainly induces luffa embryogenesis through radiation mutagenesis. Furthermore, the method improves the induction rate of embryoid by overcoming some technical bottlenecks in the culture process such as uncertain radiation dose, high contamination rate, and unsuitable culture conditions, and then forms regenerated plants, thus promoting the formation of haploid or double haploid plants and shorten the breeding years. The method can provide a technical support for luffa haploid breeding technology.

To achieve the above objective, the present disclosure adopts the following technical solutions:

The present disclosure provides a method for improving an induction rate of a luffa embryoid by radiation mutagenesis, including the following steps:
(1) conducting radiation of pollens:
subjecting pollens of a male flower to irradiation with an irradiation dose of 200 Gy to 300 Gy for 60 min;
(2) conducting pollination of radiated pollens:
subjecting resulting irradiated pollens of the male flowers to pollination on a female flower, and conducting conventional cultivation and management after the pollination is completed;
(3) conducting disinfection:
subjecting a resulting luffa fruit to disinfection by rinsing with running water and sterilization with 75% ethanol in sequence 18 d after the pollination is completed, collecting luffa seeds, and inoculating the luffa seeds into a Murashige&Skoog (MS) medium; and
(4) conducting cultivation:
transferring the luffa seeds into a tissue culture chamber to allow routine culture.

Further, the irradiation dose in step (1) is 200 Gy.

Further, the male flower is clipped in the afternoon of a day before the irradiation is conducted, and the irradiation is conducted from 8:30 to 10:00 in the morning of a next day.

Further, the female flower is clipped in the afternoon of a day of the irradiation, the pollination is conducted from 8:30 to 10:00 in the morning of a next day after the irradiation is conducted, and bagging is conducted immediately after the pollination is completed in step (2).

Further, step (3) specifically includes: rinsing a surface of the luffa fruit with the running water for 2 min to 3 min 18 d after the pollination is completed, fully absorbing water on the surface with an absorbent paper, conducting disinfection on the surface of the luffa fruit with the 75% ethanol, wiping the surface of the luffa fruit with the 75% ethanol in a sterile operating table, cutting the luffa fruit open using a sterile scalpel to separate the luffa seeds, and inoculating the luffa seeds into the MS medium.

Further, the MS medium has a pH value of 5.8 to 6.0 and includes: MS, 30 g/L sucrose, 0.2 mg/L 6-benzyladenine (6-BA), and 7 g of agar.

Further, the routine culture in step (4) is optionally conducted after heat shock culture at 32° C.

Further, step (4) specifically includes: culturing the luffa seeds inoculated into the MS medium in the dark at 32° C. for 1 d to 2 d, and then transferring the luffa seeds to the tissue culture chamber at 25° C. to allow the routine culture.

Further, the luffa is Taizhou fragrant luffa.

Beneficial effects achieved are as follows: test results have a certain relationship with the luffa varieties. The varieties that are resistant to browning must be selected, otherwise browning may easily occur during the test to affect embryo emergence.

It can be seen from the above technical solutions that compared with the prior art, the present disclosure has the following beneficial effects:

In the present disclosure, the optimal dose, lower contamination rate method, optimal culture medium formula, and culture conditions for luffa pollen radiation mutagenesis are explored by overcoming some technical bottlenecks in the culture process such as uncertainty radiation dose of luffa pollens, high contamination rate, and unsuitable culture conditions. The above conditions can significantly increase the induction rate of embryoid and promote the formation of induced regenerated plants. The haploid or double haploid plants formed by the method of the present disclosure can shorten the breeding years and provide a technical support for luffa haploid breeding technology.

### BRIEF DESCRIPTION OF DRAWINGS

To describe the technical solutions in the embodiments of the present disclosure or in the prior art more clearly, the drawings required for describing the embodiments or the prior art will be briefly described below. Apparently, the drawings in the following description merely show the embodiments of the present disclosure, and those of ordinary skill in the art can still derive other drawings from the provided drawings without creative efforts.

FIGS. 1A-1L show the influence on a viability of pollen before radiation and different radiation doses in the present disclosure, where FIGS. 1A-1C are the viability of pollen before radiation with different visual fields taken; FIGS. 1D-1F are the viability of pollen after radiation (200 Gy) with different visual fields taken; FIGS. 1G-1I are the vitality of pollen after radiation (300 Gy) with different visual fields taken; FIGS. 1J-1L are the vitality of pollen after radiation (400 Gy) with three different visual fields taken;

FIGS. 2A-2B show the influence of different disinfection methods on a contamination rate of embryoid in the present disclosure, where FIG. 2A is a first method and FIG. 2B is a second method; and

FIG. 3 shows an embryoid induced using the method in present disclosure.

### DETAILED DESCRIPTION OF THE INVENTION

The technical solutions of the embodiments of the present disclosure are clearly and completely described below with reference to the drawings in the embodiments of the present disclosure. Apparently, the described embodiments are merely a part rather than all of the embodiments of the present disclosure. All other embodiments obtained by those skilled in the art based on the embodiments of the present disclosure without creative efforts shall fall within the protection scope of the present disclosure.

In the present disclosure, the required pharmaceuticals are conventional experimental pharmaceuticals, purchased from commercial channels; the experimental methods not mentioned are conventional experimental methods and will not be described again here.

### Example 1

The variety of luffa tested was Taizhou fragrant luffa, which was provided by Taizhou Institute of Agricultural Sciences, Jiangsu Academy of Agricultural Sciences. The above test materials were sown on Jul. 15, 2021 and then planted in a plastic greenhouse at an experimental base of the Taizhou Institute of Agricultural Sciences, Jiangsu Academy of Agricultural Sciences on August 9, and sampling for pollen radiation began on October 15.

### (1) Selection of Pollen Radiation Dose

Before irradiating pollens, luffa varieties with strong growth potential were selected, and the male flowers were clipped one afternoon before flowering to prevent pollination; the clipped male flowers were collected between 8:30 and 10:00 the next morning, quickly put into a ziplock bag and then placed in a prepared ice box to ensure pollen vitality. The male flowers were immediately sent to the irradiation center for radiation. The male flowers were irradiated with γ rays with radiation doses of 200 Gy, 300 Gy, and 400 Gy separately, 60 min for each dose.

To detect pollen viability before and after irradiation, the pollen viability measurement test could be designed by referring to a process for viability measurement using the TTC method in the study of Zhang Chen et al. Specifically, a small amount of pollens from each treatment was placed on a dry glass slide, added with 1 to 2 drops of 0.5% TTC solution, mixed well, covered with a coverslip, while clean water was used as a control. After allowing to stand in a 35° C. incubator for 30 min, a microscopic examination was conducted. The pollens dyed red were the most viable, followed by light red, and colorless pollens were inactive or sterile. Three fields of view were taken from each slice, and a staining rate of pollens was counted, and the staining rate represented a percentage of pollen vitality.

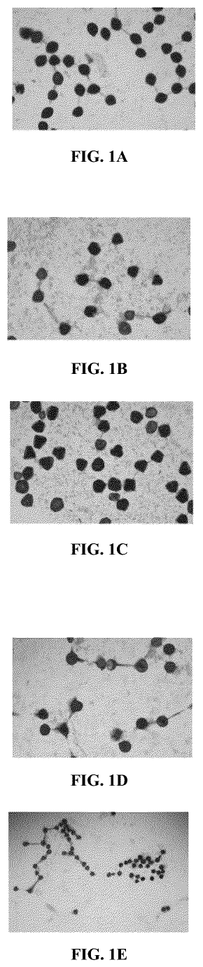

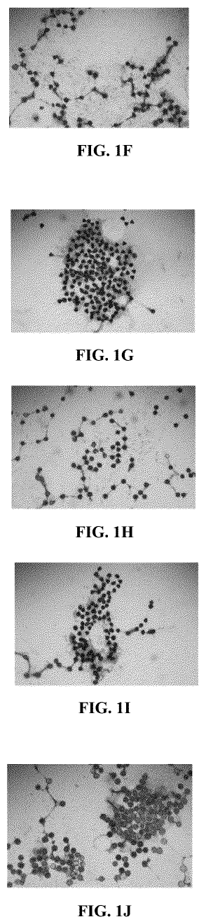

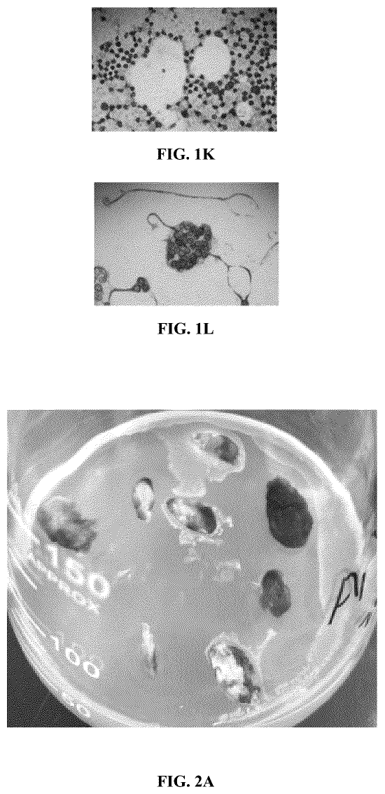

The influences of pre-irradiation and different radiation doses on pollen viability were shown in FIGS. 1A-1L.

As shown in FIGS. 1A-1DL, the luffa pollen had strong vitality before radiation and was red after staining; after 200 Gy and 300 Gy radiation, the pollen stained appeared red and had strong vitality; while after 400 Gy radiation, the pollen vitality decreased significantly.

Therefore, radiation doses of 200 Gy and 300 Gy were more appropriate to help maintain pollen vitality.

(2) Statistics on the Pollination of Irradiated Pollens and on the Fruit Setting Rate and Mature Seed Plumpness of Luffa after Irradiation

In the afternoon of the day of radiation, female flowers were clipped and pollinated with various doses of radiated pollens between 8:30 and 10:00 on the next morning. After the pollination, bagging was conducted immediately to avoid pollen contamination. After that, conventional cultivation and management was conducted. The fruit setting rate was calculated 7 d after pollination; the seeds were harvested 50 d to 55 d after pollination, and the seed plumpness was counted (Table 1).

TABLE 1
Fruit setting rate and seed plumpness of luffa
after different doses of γ-ray irradiation

| Irradiation | Pollination | Number of | Fruit setting | Mature seed |
|---|---|---|---|---|
| dose | number/seed | fruits/fruit | rate/% | plumpness/% |
| 0 | 10 | 10 | 100 | 100 |
| 200 Gy | 18 | 17 | 94.4 | 100 |
| 300 Gy | 18 | 16 | 88.9 | 92.2 |
| 400 Gy | 18 | 16 | 88.9 | 65.9 |

As shown in Table 1, the fruit setting rate and mature seed plumpness of luffa without radiation were the highest. After using 200 Gy γ-rays to irradiate luffa pollens, the fruit setting rate and mature seed plumpness of luffa were higher than those after 300 Gy and 400 Gy γ-rays; the fruit setting rate after 300 Gy γ-ray treatment was the same as that after 400 Gy γ-ray treatment, but the mature seed plumpness after 300 Gy γ-ray treatment was significantly higher than that after 400 Gy γ-ray treatment. This indicated that 200Gy γ-ray treatment had no influence on luffa seed development, while 400 Gy γ-ray treatment affected the seed development.

### (3) Influences of Different Disinfection Methods on Fruit and Seed Contamination Rates and Late-Stage Embryoid Induction Rates

18 d after pollination (when the young embryos of seeds were already full), the seeds were harvested for experimental treatment.

Two different methods were used to disinfect luffa fruits and seeds before inoculation, and the influences of the two disinfection methods on the seed contamination rate and embryoid induction rate were compared.

A first method: the surface of the luffa was rinsed with running water for 2 min to 3 min, the surface water was absorbed fully with absorbent paper, and then the surface of the fruit was disinfected with 75% ethanol before taking to a sterile operating table; the surface of the fruit was wiped with 75% alcohol on the sterile operating table, and then the luffa fruit was cut open with a sterile scalpel to separate the seeds, and the seeds were inoculated onto a prepared MS medium for culture.

A second method: the surface of the luffa was rinsed with running water for 2 min to 3 min, the surface water was absorbed fully with absorbent paper, and the luffa was placed on the sterile operating table; the luffa seeds were removed with a sterile scalpel, and then sterilized with 75% ethanol for 30 s and 7% NaClO for 10 min in sequence with shaking continuously during this period; the seeds were rinsed 3 times with sterile water, 3 to 5 min each time; the seeds were inoculated into the prepared MS medium.

The seed contamination rate was calculated after 7 d to 10 d, and the embryoid induction rate was calculated after 30 d to 35 d of culture (Table 2).

TABLE 2
Differences in disinfection of luffa seeds
by different disinfection methods

| | Number of | | Contamina- | Embryoid |
|---|---|---|---|---|
| Disinfection | seed | Number of seed | tion | induction |
| method | inoculated | contaminated | rate/% | rate/% |
| First method | 100 | 23 | 23a | 1 |
| Second method | 100 | 14 | 14b | 0 |

As shown in Table 2, disinfection using the second method could significantly reduce the contamination of luffa seeds, thus reducing the contamination rate by 39.13%, but the embryoid induction rate was 0; while the first method had a high contamination rate, but could improve the embryoid induction rate. This might be because the disinfection time and concentration of the second method caused damage to the embryos of young seeds.

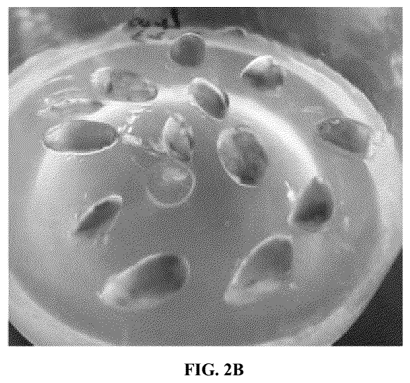

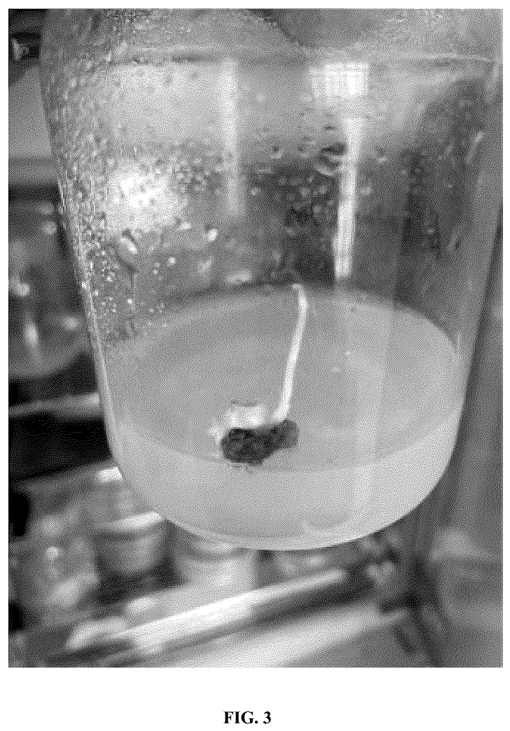

Representative photographs of the contamination rate were shown in FIG. 2A-2B.

### (4) Medium Formula

After sterilizing the luffa seeds, they were inoculated into the MS medium. After many attempts (other media tried failed to produce embryos), the formula for the medium included: MS+30 g/L sucrose+0.2 mg/L 6-BA+7 g agar, pH=5.8-6.0.

### (5) Culture Pretreatment

Embryoid induction was stimulated by low-temperature cold treatment and high-temperature heat shock treatment, with conventional treatment as a control. Specific steps were as follows.

First method: the seeds inoculated into MS medium were pretreated at a low temperature of 4° C. in the dark for 1 d and 2 d separately, and then transferred to a 25° C. culture room for culture, and the influence of low-temperature treatment was observed on the induction rate of luffa embryoid (Table 3).

Second method: the seeds inoculated into MS medium were heat-shocked at a high temperature of 32° C. in the dark for 1 d and 2 d separately, and then transferred to a 25° C. tissue culture chamber for culture, and the influence of high-temperature heat shock was observed on the induction rate of luffa embryoid (Table 3).

The callus formation of luffa was observed after 18 d to 20 d of culture, and the embryoid induction rate was calculated after 30 d to 35 d.

TABLE 3
Influences of different pretreatment cultures on callus
formation and embryoid induction rate of luffa

| | | | | Number of | |
|---|---|---|---|---|---|
| | | Number of | Number of | embryoid | Embryoid |
| | Time/ | inoculation/ | callus | induced/ | induction |
| Treatment | day | seed | formed/callus | embryoid | rate/% |
| 4° C. | 1 | 100 | 12 | 0 | 0 |
| | 2 | 100 | 18 | 0 | 0 |
| 32° C. | 1 | 100 | 29 | 1 | 1 |
| | 2 | 100 | 33 | 1 | 1 |
| 25° C. | — | 100 | 20 | 1 | 1 |

As shown in Table 3, the number of callus formed and the number of embryoid induced of luffa were the lowest after cold treatment at 4° C. for 1 d and 2 d separately; high-temperature heat shock at 32° C. was helpful for callus formation and embryoid induction in luffa. The number of callus formed after 2 d of heat shock was higher than that of 1 d of heat shock and no heat shock. However, the embryoid induction rate was the same as that without heat shock, which was 1%.

In summary, irradiation of luffa pollens with a γ-ray dose of 200 Gy can significantly increase the fruit setting rate of luffa without affecting the plumpness of mature luffa seeds. The first method for seed disinfection combined with the 32° C. heat shock pretreatment followed by routine culture can help improve the induction rate of embryoid after irradiating the pollens.

Each embodiment in the description is described in a progressive mode, each embodiment focuses on differences from other embodiments, and references can be made to each other for the same and similar parts between embodiments.

The above description of the disclosed embodiments enables those skilled in the art to achieve or use the present disclosure. Various modifications to these embodiments are readily apparent to those skilled in the art, and the generic principles defined herein may be practiced in other embodiments without departing from the spirit or scope of the present disclosure. Accordingly, the present disclosure will not be limited to these examples shown herein, but is to fall within the widest scope consistent with the principles and novel features disclosed herein.

1. A method for improving an induction rate of a luffa embryoid by radiation mutagenesis, comprising the following steps:
(1) conducting radiation of pollens:
subjecting pollens of a male flower to irradiation with an irradiation dose of 200 Gy to 300 Gy for 60 min, to obtain radiated pollens;
(2) conducting pollination of the radiated pollens:
subjecting the irradiated pollens of the male flower to pollination on a female flower, and conducting conventional cultivation and management after the pollination is completed;
(3) conducting disinfection:
rinsing a surface of a luffa fruit with running water for 2 min to 3 min 18 d after the pollination is completed, fully absorbing water on the surface with an absorbent paper, conducting disinfection on the surface of the luffa fruit with 75% ethanol, wiping the surface of the luffa fruit with the 75% ethanol in a sterile operating table, cutting the luffa fruit open using a sterile scalpel to separate luffa seeds, and inoculating the luffa seeds into a Murashige&Skoog (MS) medium; wherein
the MS medium has a pH value of 5.8 to 6.0 and comprises MS, 30 g/L sucrose, 0.2 mg/L 6-benzyladenine (6-BA), and 7 g of agar; and
(4) conducting cultivation:
transferring the luffa seeds into a tissue culture chamber to allow routine culture.

2. The method according to claim 1, wherein the irradiation dose in step (1) is 200 Gy.

3. The method according to claim 1, wherein the male flower is clipped in the afternoon of a day before the irradiation is conducted, and the irradiation is conducted from 8:30 to 10:00 in the morning of a next day.

4. The method according to claim 1, wherein the female flower is clipped in the afternoon of a day of the irradiation, the pollination is conducted from 8:30 to 10:00 in the morning of a next day after the irradiation is conducted, and bagging is conducted immediately after the pollination is completed in step (2).

5. (canceled)

6. (canceled)

7. The method according to claim 1, wherein the routine culture in step (4) is optionally conducted after heat shock culture at 32° C.

8. The method according to claim 7, wherein step (4) specifically comprises: culturing the luffa seeds inoculated into the MS medium in the dark at 32° C. for 1 d to 2 d, and then transferring the luffa seeds to the tissue culture chamber at 25° C. to allow the routine culture.

9. The method according to claim 1, wherein the luffa is Taizhou fragrant luffa.

10. The method according to claim 2, wherein the luffa is Taizhou fragrant luffa.

11. The method according to claim 3, wherein the luffa is Taizhou fragrant luffa.

12. The method according to claim 4, wherein the luffa is Taizhou fragrant luffa.

13. The method according to claim 7, wherein the luffa is Taizhou fragrant luffa.

14. The method according to claim 8, wherein the luffa is Taizhou fragrant luffa.
